(19) United States
(12) Patent Application Publication
(10) Pub. No.: US 2022/0372008 A1
(43) Pub. Date: Nov. 24, 2022

(54) LUCIFERIN DERIVATIVES AND A METHOD FOR SYNTHESIS THEREOF

(71) Applicant: VIDYASIRIMEDHI INSTITUTE OF SCIENCE AND TECHNOLOGY, Wangchan ,Rayong (TH)

(72) Inventors: Pimchai CHAIYEN, Wangchan, Rayong (TH); Pratchaya WATTHAISONG, Wangchan, Rayong (TH)

(21) Appl. No.: 17/762,060

(22) PCT Filed: Jul. 1, 2020
(86) PCT No.: PCT/TH2020/000044

§ 371 Date: Mar. 19, 2022

(30) Foreign Application Priority Data
Sep. 26, 2019 (TH) 1901006051

(51) Int. Cl. C07D 277/64

(52) U.S. Cl. CPC C07D 277/64

(57) ABSTRACT

The present disclosure relates to luciferin derivatives and method for synthesis of the luciferin derivatives. The method for synthesis of the luciferin derivatives comprises performing a first reaction by using toxic phenolic derivatives as a substrate and reacts it in buffer solutions by using thermostable dehalogenase, a group of radical scavenging enzyme, a group of polyphenol oxidase, and an FADH₂ generating system to obtain benzoquinone. The FADH₂ generating system is able to produce FADH₂ which is a substrate for the thermostable dehalogenase. Further, performing a second reaction between benzoquinone, as derived, and D-cysteine in order to obtain the luciferin derivatives having an ability to emit light at wavelengths of 600-700 nm. Therefore, the luciferin derivatives can be used in various fields such as medical research, pharmaceutical research, and other detection technologies.

### TECHNICAL FIELD

The present invention relates to luciferin derivatives and a method for synthesis of the luciferin derivatives. More specifically, the luciferin derivatives also have emission wavelengths of 600-700 nm.

### BACKGROUND

Luciferin is a natural substance found in fireflies. Luciferin is a substrate for bioluminescence reaction. The light emitted from the reaction is usually yellow-green and also visible. As a result of the special reaction between the luciferin and firefly luciferase, the bioluminescence can be utilized in various fields such as biomedical research, detection device, and food industry.

Currently, luciferin, which is generally found in fireflies, can be synthesized by chemical processes. However, it is not sufficient and does not satisfy researchers' needs, especially the needs of the medical and pharmaceutical research. These researches need new luciferin derivatives for using in complex medical researches, such as using in laboratory animals as a model to diagnose many diseases, including cancers, brain diseases, and genetic disorders, etc. Their needs are to seek for the new luciferin derivatives having luminescence activity when reacting with firefly luciferase to emit light at wavelengths greater than 600 nm, leading to the red-shifted emission wavelengths. To obtain the red light, the synthesis of luciferin derivatives and enzyme engineering of firefly luciferase are required to simultaneously modify the internal structure of the luciferase in order to produce the specificity of the reaction between luciferin derivatives and firefly luciferase. Now, the synthesis of luciferin derivatives still requires chemical reactions using transition metal catalysts and expensive substrates, while the process conditions are harsh and not environmentally friendly. Further, the product yield is not high as reported in Bioorganic & Medicinal Chemistry Letters; 2004; 2014, ChemBioChem; 2017. As a result, this synthesis of luciferin derivatives is limited for the industrial and commercial utilizations.

Journal of the American Chemical Society, JACS; 2017 has reported a chemical process for producing of new luciferin derivatives for biomedical research. However, this chemical process not only uses strong, and environmentally unfriendly chemical agents, but also the production yield is quite low. In addition, the process requires enzyme engineering of firefly luciferase by simultaneously modifying the internal structure of the enzyme in order to produce a specific reaction between luciferin derivatives and firefly luciferase. Therefore, this is a limitation for the practical use of luciferin derivatives.

From the problems and defects mentioned previously, there is an effort to develop a method for synthesis luciferin derivatives, which is able to increase luciferin derivatives production on an industrial scale. Consequently, the method is applicable at industrial level by using less expensive substrates, such as waste from chemical industrials. The synthesis method is also uncomplicated and reduces the usage of dangerous chemicals. Additionally, the novel luciferin derivatives are demanded for various medical applications and detections. Such a synthetic procedure is provided herein.

### SUMMARY

The present disclosure is directed to provide luciferin derivatives and a method for synthesis of the luciferin derivatives.

Luciferin derivatives consist of various structures, as shown in FIG. 1, wherein one or a combination of R₁, R₂, and R₃, is substituted by halogen group, nitro group, amino group, methyl group, ethyl group, and methoxy group.

In one aspect of the luciferin derivatives, the halogen groups of the luciferin derivatives groups are selected from one of fluorine, chlorine, bromine and iodine. Further, the luciferin derivatives have emission wavelengths of 600-700 nm.

A method for synthesis of luciferin derivatives comprises performing a first reaction between a substrate of phenol derivatives in a buffer solution by using a thermostable dehalogenase, a group of radical scavenging enzymes, a group of polyphenol oxidase, and a FADH₂ generating system for obtaining benzoquinone; and performing a second reaction between the benzoquinone and D-cysteine for obtaining the luciferin derivatives.

The present disclosure is aimed to synthesize the luciferin derivatives using enzymatic reactions (or biological catalysts). The substrate of a phenolic group or a phenolic derivative, are toxic agents which are normally used as for killing weeds, as herbicide, and also obtained from industries (such as from dyeing factory, fireworks and firecrackers factory, a furniture factory, etc.), resulting in being harmful to consumers of agricultural products and contaminating environment respectively. Hence, using these substrates to obtain high value luciferin derivatives is a great way to get rid of poison chemicals. In addition, the present synthesis method is not complicated and does not require harsh conditions such as high acidity or high temperatures. Furthermore, these luciferin derivatives have simple structures which can be used directly with the wild-type firefly luciferase, without requiring any enzyme engineering, leading to various and useful applications.

### BRIEF DESCRIPTION OF DRAWINGS

FIG. 1 shows the structure of the luciferin derivatives and the carbon positions on the structure.

FIG. 2 shows examples of the phenolic derivatives used as a substrate for the thermostable enzyme, HadA, for the synthesis of the luciferin derivatives.

FIG. 3 shows the multi-cycle reaction for the synthesis of the luciferin derivatives.

FIG. 4 is a graph illustrating the light absorbance of 3-bromo-4-nitrophenol degraded by the thermostable dehalogenase.

FIG. 5 is a graph illustrating the light emission at different wavelengths between standard luciferin and the luciferin derivatives obtained from the synthesis according to the present invention.

FIG. 6 shows the chemical shift of ¹H NMR of the luciferin derivatives.

FIG. 7 shows the mass analysis results of the products obtained from the method for use in the synthesis of the luciferin derivatives of the present invention

FIG. 8 is SEQ ID NO.1 indicating the amino acid sequence of the thermostable dehalogenase or HadA G513T.

### DETAILED DESCRIPTION

As described hereinafter, the present disclosure is described according to representative or preferred embodiments of the present invention and by referring to the accompanying description and drawings. However, it is to be understood the description and the drawings corresponding to such embodiments are for purpose of clarity and to aid understanding, and it is envisioned that a person having ordinary skill in the relevant art may devise various modifications without departing from the scope of the invention as defined by the appended claims.

As used herein, the terms “phenolic substance” or “phenolic family substance” or “phenolic derivative” is able to use interchangeably and broadly refers to halogenated phenols, nitro phenols, and phenols having one or more substituted groups, such as halogen groups (for example, fluorine, chlorine, bromine and iodine), nitro group, amino group, methyl group, ethyl group, and methoxy group at one or more of positions of ortho or 2, meta or 3, para or 4 (for example, p-chlorophenols (4-chlorophenols), p-bromophenols (4-bromophenols), p-iodophenols (4-iodophenols), p-fluorophenols (4-fluorophenols), p-nitrophenols (4-nitrophenols), m-fluoro-p-nitrophenol (3-fluoro-4-nitrophenol), o-amino-p-nitrophenol (2-amino-4-nitrophenol), and 2,5-difluoro-4-nitrophenol, as shown in FIG. 2.

The term “luciferin derivatives” used herein throughout the disclosure broadly refers to luciferin and/or luciferin derivatives and/or luciferin substances having substituted groups, such as one or more of halogen groups (for example, fluorine, chlorine, bromine and iodine), nitro group, amino group, methyl group, ethyl group, and methoxy group at a carbon positions 4′, 5′, 7′ of luciferin.

The term “FADH₂ generating system” used herein throughout the detail description refers to a system which capable of generating or producing FADH₂, either the system comprises FADH₂ for direct reaction, or the system comprises other reactions which can generate or produce FADH₂. For examples, the system comprises NADH, FAD and a group of flavin reductase for producing FADH₂; the system comprises G-6-PD, glucose-6-phosphate, NAD⁺, a group of flavin reductase and FAD for subsequently producing FADH₂; the system comprises GDH, glucose, NAD⁺, a group of flavin reductase and FAD for producing FADH2; the system comprises FDH, formic acid or formate , NAD⁺, a group of flavin reductase and FAD for producing FADH₂.

The term “thermostable dehalogenase” used herein refers to the dehalogenase or HadA, which is reengineered or modified, particularly modified on the surface of a wild-type dehalogenase or HadA. The reengineered or modified dehalogenase or HadA, which is called a thermostable HadA or HadA G513T, has an improvement on a temperature stability. The catalytic efficiency of the thermostable HadA or HadA G513T is in a broader range of temperature from 25-50° C. Further, the wild-type dehalogenase or HadA is a dechlorinating monooxygenase that can catalyze the elimination of nitro- and halides (F, Cl, Br, I) group from nitro- and halogenated phenols substrate. The wild type HadA can catalyze the reaction at 25° C. for 24 h. Whereas the reaction at higher temperature (>30° C.), the half-life of wild type HadA is dramatically decrease for 20 min. In accordance with various embodiments, the present disclosure relates to luciferin derivatives and a method for synthesis of the luciferin derivatives.

### LUCIFERIN DERIVATIVES

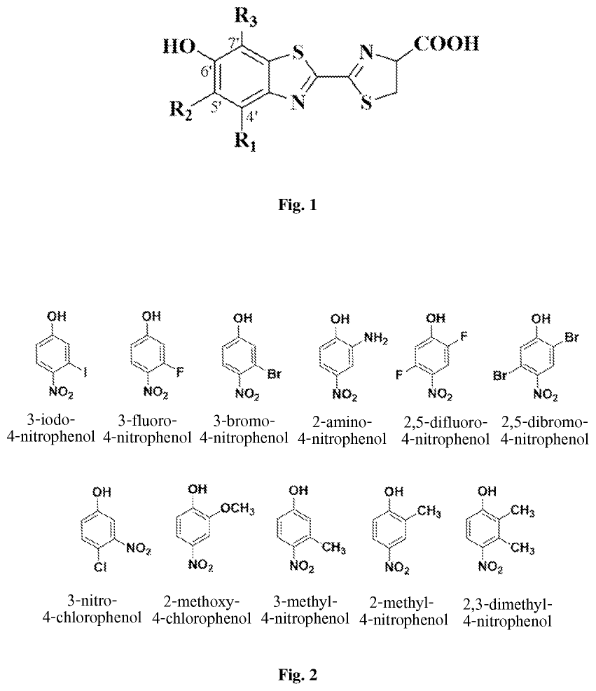

Luciferin derivatives consist of the following structure:

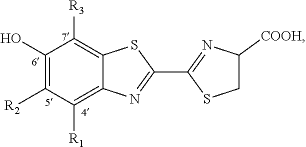

wherein one or a combination of R₁, R₂, and R₃, is substituted by halogen group, nitro group, amino group, methyl group, ethyl group, and methoxy group. (as shown in FIG. 1)

In one aspect of the luciferin derivatives, the halogen group of the luciferin derivatives is selected from one of fluorine, chlorine, bromine and iodine. Further, the luciferin derivatives have emission wavelengths of 600-700 nm.

In an aspect of the luciferin derivatives, the position Ri or R2 or R3 may be substituted by halogen group (for example, fluorine, chlorine, bromine and iodine), nitro group, amino group, methyl group, ethyl group, and methoxy group.

In another aspect of luciferin derivatives, the position R₁ and R₂, or the position R₁ and R₃, or the position R₂ and R₃ may be substituted by halogen group (for example, fluorine, chlorine, bromine and iodine), nitro group, amino group, methyl group, ethyl group, and methoxy group.

In further aspect of luciferin derivatives, all three positions R₁, R₂, and R₃ may be substituted by halogen group (for example, fluorine, chlorine, bromine and iodine), nitro group, amino group, methyl group, ethyl group, and methoxy group.

FIG. 2. shows the structures of the phenolic derivatives, which are used as substrates for the HadA G513T in order to synthesize the luciferin derivatives, wherein the phenol derivatives comprise one or more substituents such as halogen groups (for example, fluorine, chlorine, bromine and iodine), nitro group, amino group, methyl group, ethyl group, and methoxy group at one or more of positions: ortho (2), meta (3), para (4), for example, p-chlorophenols, p-bromophenols, p-iodophenols, p-fluorophenols, p-nitrophenols, m-fluoro-p-nitrophenol, m-amino-p-nitrophenol, and 2,5-difluoro-4-nitrophenol.

### A METHOD FOR SYNTHESIS OF LUCIFERIN DERIVATIVES

A method for synthesis luciferin derivatives comprises the steps of performing a first reaction between a substrate of phenol derivatives in a buffer solution by using a thermostable dehalogenase, a group of radical scavenging enzymes, a group of polyphenol oxidase, and an FADH₂ generating system for obtaining benzoquinone, wherein the FADH₂ generating system provides the FADH₂ substrate for the thermostable dehalogenase; and performing a second reaction between the benzoquinone and D-cysteine for obtaining the luciferin derivatives.

The first and the second reactions may be performed under pH of 7.0-9.0 and at a temperature range of 20-50 degrees Celsius, which this condition does not require any of high acidity and/or high temperatures.

In accordance with many embodiments, there is the step of performing a first reaction between a substrate of phenol derivatives in a buffer solution using a thermostable dehalogenase, a group of radical scavenging enzymes, a group of polyphenol oxidase, and an FADH₂ generating system for obtaining benzoquinone, wherein the FADH₂ generating system provides the FADH₂ substrate for the thermostable dehalogenase. The phenol derivatives, for example, p-chlorophenols, p-bromophenols, p-iodophenols, p-fluorophenols, p-nitrophenols, m-fluoro-p-nitrphenol, m-amino-p-nitrophenol, are suitable to be used in a range of 0.05 to 100 millimolar (mM).

The buffer solution is selected from at least one of inorganic and organic buffer solutions, wherein its usage is ranging from 1 to 1000 milliliters of volume, pH 5-9, and 20-200 mmol/l (mM) of concentration. Further, the inorganic buffer solution is selected from at least one of sodium dihydrogen phosphate solution (NaH₂PO₄), and potassium dihydrogen phosphate solution (KH₂PO₄), while the organic buffer solution is selected from at least one of HEPES solution, MOPS solution, Ammonium bicarbonate solution (NH₄HCO₃), and Ammonium formate solution (HCO₂NH₄).

The thermostable dehalogenase is a modified HadA, particularly HadA G513T (Thermostable dehalogenase; HadA G513T), containing an amino acid sequence, which is set forth in SEQ ID NO.1 as shown in FIG. 8. The thermostable dehalogenase or HadA G513T is able to catalyze in a range of temperature from 25-50° C. and its half-life at 50° C. is 200 min. However,

A concentration of thermostable dehalogenase or HadA or HadA G513T is in a range of 0.1. to 200 micromolar (μM). In addition, it is generally known that there could be modification, change, variation, elimination, replacement and/or addition of nucleotide sequences/or amino acid sequences, either codon optimization or other similar methods, resulting in that nucleotide sequences and/or amino acid sequences are suitable for each host cell species and/or able to increase the efficiency of the transcription and the translation in the host cell. Therefore, the amino acid sequences of the HadA G513T may be modified for these purposes. Moreover, the amino acid sequence of the thermostable dehalogenase or the HadA G13T may be identical at least 50% of SEQ ID NO.1. Additionally, it is also found that the HadA G13T according to the present disclosure may synthesize both of the luciferin and/or the luciferin derivatives.

The group of radical scavenging enzyme is for the removal of oxidants and free radicals destroying the stability of benzoquinone. It can be selected from one of catalase, and superoxide dismutase (SOD). A concentration of the group of radical scavenging enzymes is in a range of 0.001-200 micromolar (μM).

The group of the polyphenol oxidase is responsible for converting hydroquinone to benzoquinone by oxidation using metal and oxygen. The group of polyphenol oxidase is selected from one of tyrosinase, laccase, and peroxidase, wherein these enzymes are able to convert hydroquinone to benzoquinone by oxidation using iron (Fe²⁺), copper (Cu²⁺) and cofactor metals of these enzymes with oxygen respectively. Also, a concentration of the group of polyphenol oxidase is in a range of 0.001-200 micromolar (μ M).

The FADH₂ generating system is selected from any one of the following systems:

a first FADH₂ generating system comprising FADH₂ for a direct reaction;

a second FADH₂ generating system comprising NADH, FAD and a group of flavin reductase, wherein the NADH is a reducing agent and a substrate of a group of flavin reductase for producing FADH₂ from the FAD;

A third FADH₂ generating system comprising G-6-PD, glucose-6-phosphate, NAD⁺, a group of flavin reductase, and FAD, wherein the glucose-6-phosphate and NAD⁺ are substrates of G-6-PD for producing NADH, which is a reducing agent and a substrate of a group of flavin reductase, for subsequently producing FADH₂;

A fourth FADH₂ generating system comprising GDH, glucose, NAD⁺, a group of flavin reductase, and FAD, wherein the glucose and the NAD⁺ are substrates of the GDH for producing NADH, and further the NADH, which is a reducing agent and a substrate of a group of flavin reductase, converts the FAD to FADH₂; and

A fifth FADH₂ generating system comprising FDH, formic acid or formate , NAD⁺, a group of flavin reductase, and FAD, wherein the formic acid and the NAD⁺ are substrates of FDH for producing NADH, and then the NADH, which is a reducing agent and a substrate of a group of flavin reductase, converts the FAD to FADH₂ according to the reaction of flavin reductase.

The flavin-dependent reductase is selected from at least one of C₁, HadX, and their variants, wherein a concentration of the group of flavin reductase is in a range of 0.01-100 micromolar (μ M).

The present method for synthesis luciferin derivatives may use FADH₂ as a substrate for the thermostable dehalogenase or HadA or HadA G513T. Therefore, there may need the

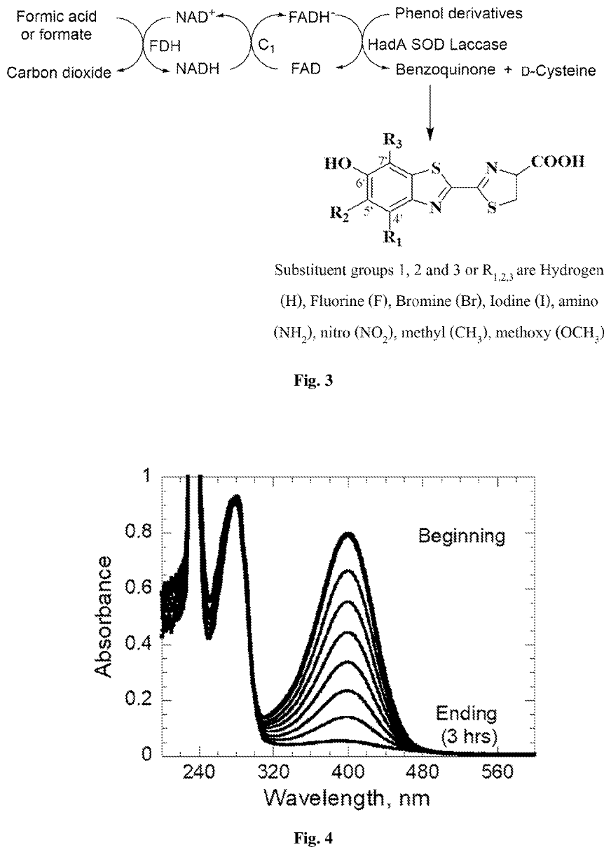

FADH₂ generating or production system in various forms, such as adding FADH₂ directly or adding a reactive substance to produce FADH₂ as shown in FIG. 3.

The group of nicotinamide adenine dinucleotide consists of NAD or NADH with the suitable amount between 1 micromolar (μ M) to 20 millimolar (mM).

For the flavin adenine dinucleotide (FAD), the amount used is in a range of 1 to 100 micromolar (μ M).

For the glucose, the glucose-6-phosphate and the formic acid or formate, the amount used in the reaction is between 0.05 millimolar (mM) to 2 Molar (M).

For the group of the dehydrogenase, such as glucose-6-phosphate dehydrogenase or G-6-PD, glucose, glucose-dehydrogenase (GDH) and formate-dehydrogenase (FDH), the amount used in the reaction is in the range of 0.1 to 20 unit per milliliter (U/ml).

In accordance with a number of embodiments, there is the step of performing a second reaction between the benzoquinone and D-cysteine for obtaining the luciferin derivatives. The amount of D-cysteine is in a range of 0.05 millimolar (mM) to 1 molar (M), while the concentration ratio between benzoquinone per D-cysteine is in the range from 1: 1-10.

FIG. 3 shows the multi-cycle reaction for obtaining the luciferin derivatives. The reaction starts from the dehydrogenase, formate dehydrogenase (FDH), reacts with the formic acid or formate, which is a substrate for FDH, and NAD (Nicotinamide adenine dinucleotide) in order to produce the reduced nicotinamide adenine dinucleotide (NADH) for the group of the flavin-dependent reductase. Here, using C₁ to catalyze the conversion of flavin adenine dinucleotide (FAD) to the reduced flavin adenine dinucleotide (reduced form) (FADH₂). After that thermostable dehalogenase, HadA G513T, takes the reduced flavin adenine dinucleotide or FADH₂ and convert the phenolic derivative or phenol group (such as halogenated phenols, nitro phenols and phenol derivatives having the substituted group of halogen, nitro, amino, ethyl and methoxy groups at one or more of the positions : ortho (2), meta (3), para, (4) to be benzoquinone derivatives (p-benzoquinone derivative), resulting in a reaction to D-cysteine and then produce the luciferin derivatives.(as shown in FIG. 1)

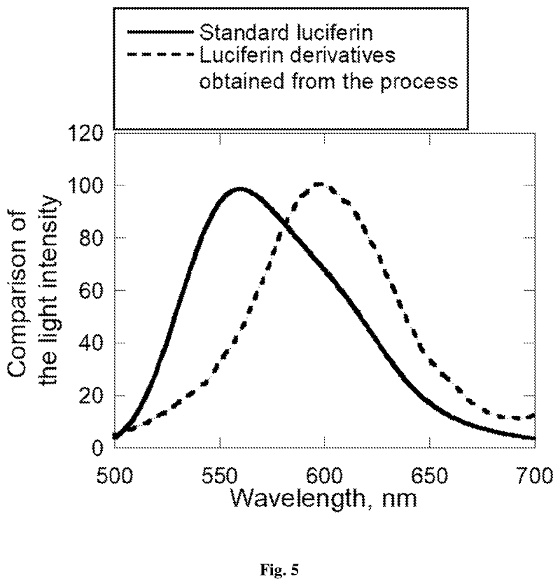

FIG. 4 is the graph showing the decrease in light absorbance at wavelengths 400-410 nm, in which the thermostable dehalogenase (HadA G513T) completely degrades the phenol derivatives (in this experiment, the 3-bromo-4-nitrophenol is used) within 300 minutes. The production yield of the luciferin derivatives is around 40-90 percent compared to the use of the phenolic derivatives.

The method for synthesis of luciferin derivatives also further comprises the step of purifying the luciferin derivatives. Further, means of purifying the luciferin derivatives is selected from at least one of organic solvent extraction, chromatography, filtration, and evaporation, leading to the purified luciferin derivatives having 50-95% purity.

FIG. 5 is the graph showing the light emitted by the reaction between the luciferase and the luciferin derivatives, which used 7′-bromoluciferin as a substrate for the firefly luciferase. The pure luciferin and the purified luciferin derivatives, which are derived from the synthesis and purification methods, reacts with the firefly luciferase, and then the emission wavelength is measured. While the standard luciferin or the luciferin obtained from this method gives the maximum emission wavelength of 560 nm, the luciferin derivatives such as 7′-bromoluciferin shows the maximum emission wavelength of 604 nm, which is red shift. This red light of the 7′-bromoluciferin is useful for medical researches (i.e. cancer detection).

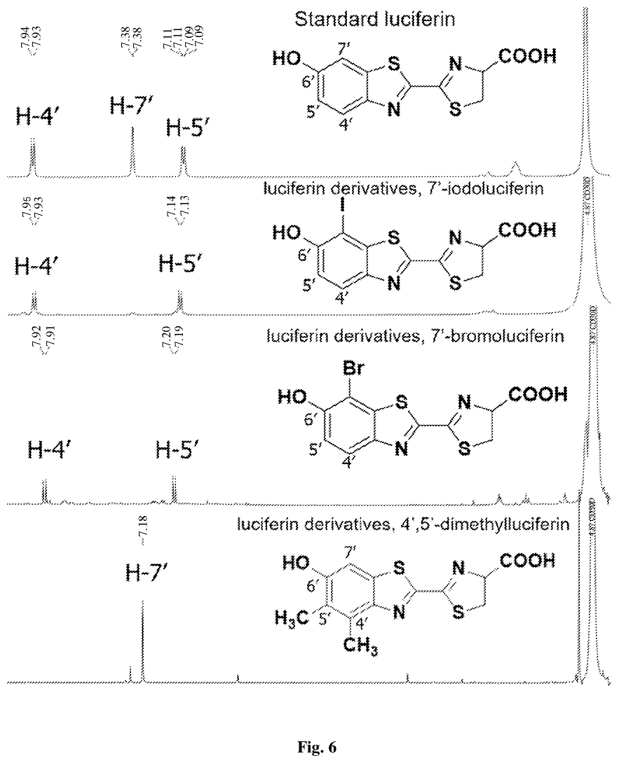

FIG. 6 is the structures and the confirmation of the phenolic derivatives derived from the present method using NMR (Nuclear magnetic resonance). The luciferin derivatives from the present method including purification, which are 7′-iodoluciferin, 7′-bromoluciferin, and 4′,5′-dimethylluciferin are compared with the standard luciferin. The standard luciferin has three chemical shift values of ¹H NMR indicating the luciferin, at 7.09 and 7.11 when expressed as doublet splitting, at 7.38 when expressed as singlet splitting 7.93, and 7.94 when expressed as doublet splitting. The luciferin derivatives substituted with halogen atom at the position 7′ of the luciferin, Iodine (I) and Bromine (Br), have the chemical shifts appear at position 7.38 and also the chemical shift values of ¹H NMR at other positions are slightly different from the standard (or natural) luciferin. The luciferin derivative substituted with methyl group at the position 4′ and 5′ of the luciferin has a chemical shift appears at position 7.18 (singlet splitting). The ¹H NMR chemical shift of 4′,5′-dimethylluciferin is different from ¹H NMR of standard luciferin at position 7.38.

### PROPERTIES OF THE LUCIFERIN DERIVATIVES OF THE PRESENT DISCLOSURE

1. Bioluminescence/Luminescence of the luciferin derivatives when reacting with the firefly luciferase

The reaction between the luciferin derivatives of the present disclosure and the firefly luciferase can emit the light. By using a spectrofluorometer under bioluminescent mode, the emission wavelength can be measured for proof of the bioluminescence property, whether the luciferin derivatives are able to be substrates for the firefly luciferase or not.

The experiment shows that the standard luciferin and the luciferin derivatives (i.e. 7′-bromoluciferin) are able to be a substrate for the firefly luciferase by emitting the wavelengths of 560 nm and 604 nm respectively.

2. Molecular mass analysis using QTOF-mass spectrometer

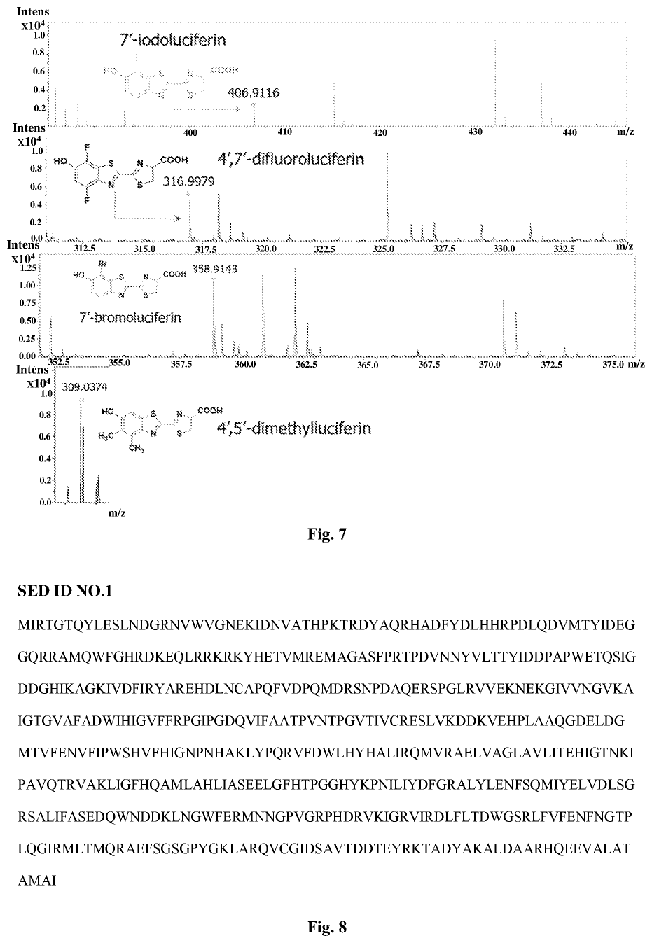

By using QTOF-mass spectrometer, the molecular mass of the synthesized luciferin derivatives, 7′-iodoluciferin (C₁₁H₇IN₂O₃S₂), 4,7′-difluoroluciferin (C₁₁H₆F₂N₂O₃S₂), and 7′-bromoluciferin (C₁₁H₇BrN₂O₃S₂), and 4′,5′-dimethylluciferin (C₁₃H₁₂N₂O₃S₂) are 406.9116, 316.9979, 358.9143, and 309.0374, respectively, which indicate the molecular mass of the expected compounds, shown in FIG. 7.

### Example 1: The synthesis of 7′-bromoluciferin from 3-bromo-4-nitrophenol

Substrates for synthesis of the luciferin derivatives 7′-bromoluciferin are:
0 2 millimolar 3-bromo-4-nitrophenol
100 millimolar HEPES buffer solution
50 micromolar thermostable dehalogenase (HadA G513T)
2.0 micromolar SOD
2.0 micromolar laccase
a fifth FADH₂ generating system comprises 2.0 micromolar FDH, 20 millimolar formic acid, 10.0 micromolar NAD⁺, flavin-dependent reductase used in example 1 is 2.0 micromolar C₁, and 4.0 micromolar FAD
2 millimolar D-cysteine

All the solutions are mixed together, at pH of 8.0, 5 ml, in a container and stirred with a magnetic bar at a temperature of 35° C. The reaction occurs when the thermostable dehalogenase solution is added to the container, then allowing the reaction to complete for about 200 minutes. Then, the luciferin derivatives are purified by pushing the solution via a nitrogen gas through the membrane filter (stirred-cell) with a cut-off value of 10 kDa in combination with the extraction by organic solvents, i.e. ethyl acetate. The solution after extraction with ethyl acetate, will be analyzed for purity and purification again with HPLC by tracking the light absorbance at a wavelength of 327 nm. The solution is separated through a non-polar column (Reverse phase C18) using a mixture of water and methanol with the addition of 0.1% v/v formic acid, as elution mobile phase. The luciferin derivatives will be collected and then freeze-dried. The purified luciferin derivatives are then produced for 2 milligrams.

### Example 2: The synthesis of 4′,7′-difluoroluciferin from 2,5- difluoro-4-nitrophenol

Substrates for synthesis of the luciferin derivatives 4′,7′-difluoroluciferin are:
1.0 millimolar 2,5-difluoro-4-nitrophenol
100 millimolar MOPS buffer solution
50 micromolar HadA G513T
2.0 micromolar SOD
2.0 micromolar laccase
a fourth FADH₂ generating system comprises 4.0 micromolar GDH, 20.0 millimolar glucose, 10.0 micromolar NAD⁺, 2.0 micromolar flavin-dependent reductase (HadX), 4.0 micromolar FAD, and 2.0 millimolar D-cysteine

All the solutions are mixed together, at pH of 8.0, 5 ml, in a container and stirred with a magnetic bar at a temperature of 35° C. The reaction occurs when thermostable dehalogenase solution or HadA G513T is added to the container, then allowing the reaction to complete. The luciferin derivative 4′,7′-difluoroluciferin prepared from 2,5-difluoro-4-nitrophenol will be finally obtained.

### Example 3: The synthesis of 4′,5′-dimethylluciferin from 2,3- dimethyl-4-nitrophenol

Substrates for synthesis of the luciferin derivative 4′,5′-dimethylluciferin are:
0.5 millimolar 2,3-dimethyl-4-nitrophenol
100 millimolar sodium phosphate buffer solution
100 micromolar thermostable dehalogenase (HadA G513T)
5.0 micromolar SOD
2.0 micromolar tyrosinase
a second FADH₂ generation system comprising NADH and FAD, wherein the NADH is a reducing agent and a substrate of a group of flavin reductase for producing FADH₂ from the FAD
5 millimolar D-cysteine

All of the solutions are mixed together, at pH of 7.5, 20 ml, in a container and stirred with a magnetic bar at a temperature of 35° C. The reaction occurs when thermostable dehalogenase solution or HadA G513T is added to the container, then allowing the reaction to complete. The luciferin derivative 4′,5′-dimethylluciferin prepared from 2,3-dimethyl-4-nitrophenol will be finally obtained.

1-18 (canceled)

19. A luciferin derivative consisting of the following structure: 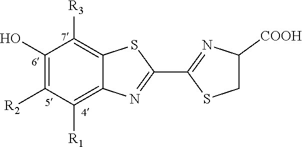
wherein the luciferin derivative comprises one of R₁, R₂, and R₃ or a combination of R₁, R₂, or R₃, substituted by a halogen group, a nitro group, an amino group, a methyl group, an ethyl group, or a methoxy group.

20. The luciferin derivative according to claim 19, wherein the halogen group is selected from one of fluorine, chlorine, bromine and iodine.

21. The luciferin derivative according to claim 19, wherein the R₁ and R₂ are substituted by a methyl.

22. The luciferin derivative according to claim 19, wherein the R₂ and R₃ are substituted by an iodine or a bromine.

23. The luciferin derivative of claim 19, wherein the R₂ is substituted by a methyl.

24. The luciferin derivative according to claim 19, wherein the luciferin derivatives have emission wavelengths of 600-700 nm.

25. A method for synthesis of a luciferin derivative of claim 19 comprising:
obtaining benzoquinone by performing a first reaction in a buffer solution using substrates of phenol derivatives, thermostable dehalogenases, a group of radical scavenging enzymes, a group of polyphenol oxidases, and an FADH₂ generating system; and
performing a second reaction between the benzoquinone and D-cysteine for obtaining the luciferin derivative.

26. The method according to claim 25, wherein the thermostable dehalogenase is HadA G513T.

27. The method according to any one of claim 25, wherein the thermostable dehalogenase has an amino acid sequence corresponding to at least 50% of SEQ ID NO.1.

28. The method according to claim 25, wherein the group of radical scavenging enzymes is selected from one of catalase, and superoxide dismutase.

29. The method according to claim 25, wherein the group of polyphenol oxidases is selected from one of tyrosinase, laccase, and peroxidase.

30. The method according to claim 25, wherein the substrates of phenol derivatives are any one or any combination of 3-iodo-4-nitrophenol, 3-fluoro-4-nitrophenol, 3-bromo-4-nitrophenol, 2-amino-4-nitrophenol, 2,5-difluoro-4-nitrophenol, 2,5-dibromo-4-nitrophenol, 3-nitro-4-chlorophenol, 2-methoxy-4-chlorophenol, 3-methyl-4-nitrophenol, 2-methyl-4-nitrophenol and 2,3-dimethly-4-nitrophenol.

31. The method according to claim 25, wherein the FADH2 generating system is selected from one of
a first FADH₂ generating system comprising FADH₂ for a direct reaction;
a second FADH₂ generating system comprising NADH, FAD and a group of flavin reductases, wherein the NADH is a reducing agent and a substrate of a group of flavin reductases for producing FADH₂ from the FAD;
A third FADH₂ generating system comprising G-6-PD, glucose-6-phosphate, NAD⁺, a group of flavin reductases, and FAD, wherein the glucose-6-phosphate and NAD⁺ are substrates of G-6-PD for producing NADH, which is a reducing agent and a substrate of a group of flavin reductases, for subsequently producing FADH2;
A fourth FADH₂ generating system comprising GDH, glucose, NAD⁺, a group of flavin reductases, and FAD, wherein the glucose and the NAD are substrates of the GDH for producing NADH, and further the NADH, which is a reducing agent and a substrate of a group of flavin reductases, converts the FAD to FADH₂; and
A fifth FADH₂ generating system comprising FDH, formic acid/or formate, NAD⁺, a group of flavin reductases, and FAD, wherein the formic acid and the NAD are substrates of FDH for producing NADH, and then the NADH, which is a reducing agent and a substrate of a group of flavin reductases, converts the FAD to FADH₂.

32. The method according to claim 31, wherein the group of flavin reductase is selected from at least C1 and HadX.

33. The method according to claim 25, wherein the reacting step is performed under pH of 7.0-9.0 and at a temperature range of 20-50 degrees Celsius.

34. The method according to claim 25 further comprising purifying the luciferin derivatives.


SEQUENCE LISTING (1 sequence)

SEQ ID NO: 1 - protein, 517 aa, Escherichia coli
MIRTGTQYLESLNDGRNVWVGNEKIDNVATHPKTRDYAQRHADFYDLHHRPDLQDVMTYIDEGGQRRAMQWFGHRDKEQLRRKRKYHETVMREMAGASFPRTPDVNNYVLTTYIDDPAPWETQSIGDDGHIKAGKIVDFIRYAREHDLNCAPQFVDPQMDRSNPDAQERSPGLRVVEKNEKGIVVNGVKAIGTGVAFADWIHIGVFFRPGIPGDQVIFAATPVNTPGVTIVCRESLVKDDKVEHPLAAQGDELDGMTVFENVFIPWSHVFHIGNPNHAKLYPQRVFDWLHYHALIRQMVRAELVAGLAVLITEHIGTNKIPAVQTRVAKLIGFHQAMLAHLIASEELGFHTPGGHYKPNILIYDFGRALYLENFSQMIYELVDLSGRSALIFASEDQWNDDKLNGWFERMNNGPVGRPHDRVKIGRVIRDLFLTDWGSRLFVFENFNGTPLQGIRMLTMQRAEFSGSGPYGKLARQVCGIDSAVTDDTEYRKTADYAKALDAARHQEEVALATAMAI